(19) United States
(12) Patent Application Publication
(10) Pub. No.: US 2016/0075996 A1
(43) Pub. Date: Mar. 17, 2016

(54) METHOD FOR PRODUCING NK CELL-ENRICHED BLOOD PREPARATION

(71) Applicant: BIOTHERAPY INSTITUTE OF JAPAN, Tokyo (JP)

(72) Inventors: Hiroshi TERUNUMA, Tokyo (JP); Xuewen DENG, Chengdu (CN); Mie NIEDA, Tokyo (JP)

(21) Appl. No.: 14/780,394

(22) PCT Filed: Mar. 27, 2013
(86) PCT No.: PCT/JP2013/059037

§ 371 Date: Sep. 25, 2015

(51) Int. Cl. C12N 5/0783; A61K 35/17

(52) U.S. Cl. CPC C12N 5/0646; A61K 35/17; C12N 2501/20; C12N 2501/599; C12N 2502/1164; C12N 2523/00

(57) ABSTRACT

It is intended to provide a method for producing an NK cell-enriched blood preparation, which is low invasive and is capable of conveniently and rapidly growing NK cells, etc. in blood collected from an organism. The NK cells in blood are stimulated with NK cell growth-stimulating factors comprising an anti-CD16 antibody, OK432, an anti-CD137 antibody, and a cytokine. Then, the blood is cultured at a physiological cell temperature to produce an NK cell-enriched blood preparation.

### TECHNICAL FIELD

The present invention relates to a method for producing a blood preparation containing activated and grown NK cells, a blood preparation produced by the method, and a composition for NK cell activation.

### BACKGROUND ART

Cancer, also known as malignant neoplasm, has been the leading cause of death in Japanese since 1981 and accounts for approximately 30% of all deaths. Although medical advances have drastically improved its cure rate and survival rate, cancer is still an intractable disease. Standard treatment methods for cancer are surgical therapy, chemotherapy, and radiotherapy. In recent years, immunotherapy has received attention as a novel treatment method, and various methods have been developed so far (Non Patent Literature 1). The immunotherapy refers to a method for treating cancer, viral infection, or the like by body's immunity. Examples thereof include cytokine therapy, vaccinotherapy, BRM (biological response modifier) therapy, and cellular immunotherapy.

The cytokine therapy refers to a treatment method involving directly administering, into an organism, cytokines having the effect of growing or activating lymphocytes such as T cells or NK cells to thereby kill cancer cells or virus-infected cells. This corresponds to, for example, a treatment method based on the administration of interleukin 2 (IL-2) or interferon (Non Patent Literature 2). Unfortunately, this treatment method has failed to produce expected outcomes in clinical trials and causes undesired serious adverse reaction such as organ dysfunction or fluid retention (in the case of IL-2 administration), or cold symptoms or mental disorder (in the case of interferon administration).

The vaccinotherapy refers to a treatment method involving direct or indirect inoculation with a cancer cell-specific antigen or peptide to activate the immune system against the antigen (Non Patent Literature 3). This treatment method has been reported to be effective for some cases, but is disadvantageously ineffective for tumor or the like without HLA class I expressed therein.

The BRM therapy refers to a treatment method using a substance modifying the biological response of patients to tumor cells or the like (Non Patent Literature 4). For example, PSK, bestatin, and OK432 are known as BRM. This treatment method, albeit with proven efficacy on some cancers, etc., is more likely to be a supportive therapy that produces effects when used in combination with surgical therapy or other treatment methods such as chemotherapy, which lowers immunity. In addition, this treatment method does not always fortify immunity and, unfortunately, its own anticancer effect or the like is weak.

The cellular immunotherapy refers to a treatment method involving subjecting immunocytes collected from a patient to ex vivo treatment such as activation or growth and then bringing these cells back to patient's body to enhance the immunity of the patient, and is also called “adoptive immunotherapy (adoptive immunotherapy in the broad sense)” (Non Patent Literature 5). The cellular immunotherapy is classified into activated lymphocyte therapy and dendritic cell therapy depending on the type of immunocytes treated ex vivo. Of them, the dendritic cell therapy has just entered the clinical stage and thus, has not yet produced sufficient results in clinical trials to determine its efficacy.

The activated lymphocyte therapy is further classified into: activated lymphocyte therapy in the narrow sense, which involves activating or growing T cells ex vivo (activated T lymphocyte therapy or adoptive immunotherapy in the narrow sense); and activated NK cell therapy, which involves activating or growing NK cells.

The activated lymphocyte therapy in the narrow sense corresponds to, for example, LAK (lymphokine-activated killer cell) therapy, TIL (tumor-infiltrating lymphocyte) therapy, and CTL (cytotoxic T lymphocytes) therapy.

The LAK therapy refers to a method involving lymphocytes collected from a patient, activating or growing T cells or NK cells by culture, and then bringing these cells back to patient's body (Non Patent Literature 6). This method requires administering a large amount of IL-2 into an organism for maintaining the LAK activity administered into the organism, resulting in undesired adverse reaction, as in the IL-2-based cytokine therapy, or less-than-expected effects.

The TIL therapy refers to a method involving collecting lymphocytes infiltrated into tumor cells or the like, culturing them ex vivo as in the LAK therapy, and then bringing them back to the body (Non Patent Literature 7). Unfortunately, for this method, surgically excised tissues are only way to collect lymphocytes, and this method produces less-than-expected effects.

The CTL therapy refers to a method involving stimulating lymphocytes by coculture with cancer cells or the like collected by surgery to induce lymphocytes specific for the cancer cells or the like (Non Patent Literature 8). This method has been reported to be effective for some cases, but is very highly invasive and applicable to only limited cases because cancer cells must be collected by surgery. The further problems thereof are, for example: treatment is difficult to achieve if cancer cells can be neither collected nor cultured; and this method is effective only for cancer expressing major histocompatibility antigens.

Meanwhile, the activated NK cell therapy refers to a method involving bringing grown and activated NK cells back into the body. NK cells are a population of lymphocytes capable of killing cancer cells or virus-infected cells without being sensitized to antigens (Non Patent Literatures 9 to 11). The NK cells are known to be capable of suppressing cancer infiltration or metastasis in animal experiments (Non Patent Literature 12). According to long-term large-scale cohort study, it has been reported that cancer occurs with a significantly low incidence in humans having highly active NK cells in peripheral blood compared with humans having low active NK cells in peripheral blood (Non Patent Literature 13). Hence, NK cells from a patient can be grown and activated in large amounts ex vivo and then brought back into patient's body to thereby treat cancer, viral infection, or the like in the patient. However, NK cells are usually found to make up only a small percent to a dozen percent of lymphocytes even in healthy individuals, and the number of NK cells is further reduced in the case of cancer patients. Moreover, NK cells in blood often exhibit lower cytotoxic activity against cancer cells in cancer patients than in healthy individuals even when the same numbers of NK cells are present therein. Thus, growth and activation by culture are absolutely necessary for the therapy. NK cells had been considered difficult to grow ex vivo. Nevertheless, many studies have reported in recent years that NK cells were successfully grown and cultured (Non Patent Literatures 14 to 17). These methods, however, utilize cancer cells cultured for NK cell enrichment or transformed cells and thus, have not yet overcome problems associated with safety in clinical application or practicality. Also, NK cell growth efficiency and cell activity have been at the less-than-satisfactory level.

As described above, all the conventional immunotherapy methods have presented insufficient therapeutic effects, serious adverse reaction, or other possible improvements thereto.

Thus, as a result of conducting diligent studies to solve these problems, the inventors of the present application had successfully developed a method for producing an NK cell-enriched blood preparation, which is capable of efficiently enriching NK cells in blood collected from an organism, by treatment together with NK cell growth-stimulating factors at a particular temperature for a particular time, and received a patent for the production method and the NK cell-enriched blood preparation (Patent Literature 1). The NK cell-enriched blood preparation obtained by this method has been used actually in the clinical stage to produce a large number of very favorable clinical outcomes (Non Patent Literatures 18 to 20). This production method, however, has a slightly complicated production process in which a medium must be kept at a particular temperature for a relatively long time (10 to 30 hours) for the sufficient activation of NK cells. In addition, this method requires laborious temperature control and much time to complete the preparation.

### CITATION LIST

### Patent Literature

Patent Literature 1: JP Patent No. 4275680

### Non Patent Literature

Non Patent Literature 1: Milani V, et al., 2009, J trans Res, 7 (50): 1-18.
Non Patent Literature 2: Rosenberg S A, et al., 1985, J Exp Med., 161: 1169-88.
Non Patent Literature 3: Bendandi, M. et al., 1999, Nature Med, 5: 1171-1177.
Non Patent Literature 4: Fisher M, et al., 2002, Anticancer Res., 22: 1737-54.
Non Patent Literature 5: Takayama Y et al., 2000, Lancet, 356: 802-807.
Non Patent Literature 6: Mule J J, et al., 1985, J Immunol., 135: 646-52.
Non Patent Literature 7: Dudley M E, et al., 2003, J Immunother., 26: 332-42.
Non Patent Literature 8: Araki K et al., 2000, Int J Oncol., 17 (6): 1107-18.
Non Patent Literature 9: Stagg J and Smyth M J., 2007, Drug News Perspect, 20 (3): 155-163.
Non Patent Literature 10: Terme M et al., 2008 Nat. Immunol, 9 (5): 486-493.
Non Patent Literature 11: Vivier E et al., 2008, Nat. Immunol, 9 (5): 503-510.
Non Patent Literature 12: Dewan M Z et al., 2007, Breast Cancer Res Treat, 104: 267-275.
Non Patent Literature 13: Imai K et al., 2000, Lancet, 356: 1795-1799.
Non Patent Literature 14: Harada H et al., 2002, JPN J Cancer Res, 93: 313-9.
Non Patent Literature 15: Carlens S et al., 2001 Hum Immunol, 62: 1092-8.
Non Patent Literature 16: Berg M et al., 2009, Cytotherapy, 11 (3): 341-55.
Non Patent Literature 17: Fujisaki H et al., 2009, Cancer Res, 9: 4010-7.
Non Patent Literature 18: Brillard E et al., 2007, Exp Hemato, 35: 416-425.
Non Patent Literature 19: Cooke A and Brode S., 2008, Critical Rev Immnunol., 28 (2): 109-126.
Non Patent Literature 20: Hsu K C et al., 2005, Blood, 105: 4878-4884.

### SUMMARY OF THE INVENTION

### Technical Problem

An object of the present invention is to develop a novel method for producing an NK cell-enriched blood preparation, which is low invasive to donors and patients and is capable of rapidly enriching NK cells in blood collected from an organism in large amounts by a convenient production process, and to provide an NK cell-enriched blood preparation obtained by the method in a safe and relatively inexpensive manner.

### Solution to Problem

In order to attain the object, the present inventors have conducted further studies on a method for producing an NK cell-enriched blood preparation, and consequently successfully developed a novel method for producing an NK cell-enriched blood preparation which enhances NK cell growth activity without the need of an essential step comprising keeping cells at a particular temperature for a particular time in the method for producing an NK cell-enriched blood preparation according to JP Patent No. 4275680.

The present invention has been completed on the basis of these development results and provides the followings:

(1) A method for producing an NK cell-enriched blood preparation, comprising:

a stimulation step of stimulating NK cells contained in blood collected from an organism, with NK cell growth-stimulating factors comprising an anti-CD16 antibody, OK432, an anti-CD137 antibody, and a cytokine; and a culture step of culturing the blood at a physiological cell temperature after the stimulation step.

(2) The production method according to (1), wherein the cytokine is IL-2.

(3) The production method according to (1) or (2), wherein the NK cell growth-stimulating factors further comprise an anti-CD3 antibody, and/or a bisphosphonate derivative or a salt thereof, or a hydrate thereof.

(4) The production method according to any of (1) to (3), wherein the physiological cell temperature is 36.5 to 37.5° C.

(5) The production method according to any of (1) to (4), wherein the culture period in the culture step is 7 days to 30 days.

(6) The production method according to any of (1) to (5), wherein the anti-CD16 antibody is immobilized on a solid-phase support.

(7) An NK cell-enriched blood preparation obtained by a production method according to any of (1) to (6).

(8) A composition for NK cell enrichment comprising an anti-CD16 antibody, OK432, an anti-CD137 antibody, and a cytokine.

(9) The composition according to (8), wherein the cytokine is IL-2.

(10) The composition according to (8) or (9), further comprising an anti-CD3 antibody and/or a bisphosphonate derivative or a salt thereof, or a hydrate thereof.

(11) A kit for production of NK cell-enriched blood comprising a composition for NK cell enrichment according to any of (8) to (10).

### Advantageous Effects of the Invention

The method for producing an NK cell-enriched blood preparation according to the present invention can prepare NK cells in blood more rapidly and more conveniently at a more improved growth rate of NK cells than conventional methods. Moreover, the production method of the present invention is capable of production from peripheral blood and is thus advantageously low invasive to donors and patients.

### BRIEF DESCRIPTION OF THE DRAWINGS

FIG. 1 shows a cytogram at culture day 0, day 14 and day 21 for samples a (upper) and b (lower) in Examples. In each cytogram, the abscissa represents the fluorescence intensity of a PC5-labeled anti-CD3 antibody (day 0) or an ECD-labeled anti-CD3 antibody (days 14 and 21) on a log scale. The ordinate represents the fluorescence intensity of a PE-labeled anti-CD56 antibody (day 0) or a PC5-labeled anti-CD56 antibody (days 14 and 21) on a log scale. The cytogram is divided into four zones (B1 to B4) based on these various fluorescence intensities. NK cells are distributed in zone B1 (CD3⁻CD56⁺); T lymphocytes are distributed in zones B2 (CD3⁺CD56⁺) and B4 (CD3⁺CD56⁻); and the other cells, such as B cells, are distributed in zone B3 (CD3⁻CD56⁻). The numeric value in each fraction represents the ratio (%) of the cells contained in the fraction to all the assayed cultured cells.

FIG. 2 is a cell growth curve showing the relationship between the number of culture days and the total number of cultured cells for samples a and b of Examples.

FIG. 3 shows the cytotoxic activity of NK cells at culture day 14 and day 21 for samples a and b in Examples. The E/T ratio shown in the X-axis is the ratio between cultured NK cells used as effector cells (E) and target K562 cells (target cells: T). The Y-axis shows a relative value (%) of the cytotoxic activity of NK cells compared with K562 to a control before cytotoxicity without the addition of effector cells.

FIG. 4 is a cell growth curve showing the relationship between the number of culture days and the total number of cultured cells for samples α and β in Examples.

### DETAILED DESCRIPTION OF THE PREFERRED EMBODIMENTS

### 1. Method for Producing NK Cell-Enriched Blood Preparation

1-1. Summary

The first aspect of the present invention relates to a method for producing an NK cell-enriched blood preparation. The feature of this aspect is that NK cells in blood collected from an organism are stimulated with growth-stimulating factors, and then, the blood is cultured at a physiological cell temperature.

In the present invention, the term “enrichment” means to grow and/or activate cells or to have grown and/or activated cells. In the present specification, the term “activation” means to enhance or potentiate functions possessed by cells, particularly, NK cells. Examples thereof include to enhance or potentiate cytotoxic function and the expression of NK cell surface receptors involved in activity and/or growth. In the present invention, the “NK cell-enriched blood preparation” refers to a preparation mainly composed of blood containing a large number of activated NK cells obtained by the production method of this aspect.

1-2. Constitution

The method for producing an NK cell-enriched blood preparation according to this aspect comprises a stimulation step and a culture step. Hereinafter, the constitution of each step will be described specifically. This aspect is based on the premise that, as a rule, each operation employs already sterilized reagents, media, tools, etc., and culture is performed in a sterile environment such as a clean bench in a clean room. This is because contamination with bacteria or the like is prevented.

1-2-1. Stimulation Step

The “stimulation step” is the step of stimulating NK cells contained in blood collected from an organism with NK cell growth-stimulating factors.

(1) Blood

In the present invention, the “blood” refers to a blood component containing NK cells. The blood (component) corresponds to, for example, whole blood, cord blood, bone marrow fluid, or a portion of its components, for example, mononuclear cells. Any blood (component) can be used, and mononuclear cells are preferable because blood components such as erythrocytes or granulocytes might become a hindrance to the production of the NK cell-enriched blood preparation. Among others, peripheral blood mononuclear cells (hereinafter, referred to as PBMCs) obtained from peripheral blood are particularly preferable. This is because peripheral blood can be collected easily from organisms at any time with procedures low invasive to the donors.

In the present invention, the “organism” refers to a living mammal. The type of the mammal is not particularly limited, and a human is preferable. The donor organism is desirably of the same type as in a mammal that receives the NK cell-enriched blood preparation obtained by the production method of the present invention. For example, when the NK cell-enriched blood preparation of the present invention is administered to a human, blood is preferably collected from a human. More preferably, blood is collected from a donor having an HLA (human leucocyte antigen) genotype compatible with that of a recipient. For example, when the recipient has underwent organ transplantation or stem cell transplantation, the donor usually corresponds to a donor of this organ or the stem cells. In most cases, a preferable recipient having a compatible HLA genotype is a blood relative. A blood preparation derived from the blood of a donor having a compatible HLA genotype can minimize the possibility of rejection in the recipient after administration. Thus, the donor is most preferably a recipient itself that receives the NK cell-enriched blood preparation of the present invention, i.e., blood collection is predicated on adoptive immunotherapy. When blood collection is predicated on adoptive immunotherapy, the donor organism does not have to be healthy. For example, blood can be collected even from a donor having cancer or viral infection. In the present invention, the adoptive immunotherapy in the description below means the adoptive immunotherapy in the broad sense described above, unless otherwise specified.

The phrase “collected from an organism” means derived from an organism. Possible collected blood is, for example, peripheral blood or bone marrow fluid collected by the direct insertion of an injection needle or the like to the organism, cord blood collected directly from the postpartum umbilical cord, or a perfusate of a transplanted organ. The collected blood may be blood obtained by adding heparin or the like for anticoagulation treatment to the collected blood or further isolating mononuclear cells therefrom and then temporarily refrigerating or cryopreserving it, followed by collection.

(2) Preparation of Blood

When the blood used in this step is directly collected from the organism, a collection method therefor can follow a blood collection method known in the art. For example, peripheral blood may be collected by injection to the peripheral vein or the like; bone marrow fluid may be collected by bone marrow aspiration; and cord blood may be collected by the injection of a needle to the postpartum umbilical cord before placenta delivery. Hereinafter, the collection of peripheral blood will be described specifically with reference to one example.

Peripheral whole blood can be collected according to a whole blood collection method known in the art using a vacuum blood collection tube, a blood collection bag or the like by the insertion of an injection needle to the peripheral blood vessel, for example, the vein or artery, of the organism. The volume of blood collected varies depending on the necessary amount of the NK cell-enriched blood preparation. Usually, 20 mL to 60 mL suffices for the blood preparation produced, for example, for a single dose to an adult. However, the number of PBMCs in blood may be extremely reduced, for example, in cancer patients. In such a case, only PBMCs may be collected selectively in a necessary amount by apheresis. For preventing the coagulation of the blood thus collected, it is preferred to coat in advance, for example, the inside of a blood collection tube syringe, with an anticoagulant such as heparin or a blood coagulation inhibitor, or to add heparin or the like to the collected blood. Alternatively, plasma is separated from the peripheral whole blood, and only the remaining blood cell components may be used in the present invention. The plasma separation can be achieved, for example, by the centrifugation at 2000 rpm to 4000 rpm for 5 to 20 minutes of peripheral whole blood transferred to a centrifuge tube, followed by the removal of the supernatant. The separated plasma can be inactivated by heating at 56° C. for approximately 30 minutes, then centrifuged at 2000 rpm to 4000 rpm for 5 to 20 minutes, and also used as nutrients for cell culture by the removal of precipitates such as platelet.

PBMCs may be further separated, if necessary, from the peripheral whole blood. PBMCs can be obtained from peripheral whole blood or from blood cell components after plasma separation using a density-gradient centrifugation method with Ficoll-Hypaque or Ficoll-Conray as a specific gravity solution. A commercially available separating solution or the like can be used conveniently as such a specific gravity solution. For example, Ficoll-Paque PLUS (GE Healthcare Life Sciences Corp.) or LYMPHOPREP (AXIS-SHIELD plc) can be used. A method for separating PBMCs can follow the protocol supplied with the kit.

The PBMCs thus separated are washed several times with PBS (−) or a medium for cultured cells to remove the specific gravity solution. In this context, for example, serum-free PBS (−) or a RPMI-1640 medium, or a serum-free medium for use in other culture can be used as the medium for cultured cells. After the washing with this PBS (−) or medium, it is preferred to count the number of collected PBMCs using a hemacytometer. In the case of a healthy human adult, usually, 2×10⁷ or more PBMCs can be collected from 20 mL to 60 mL of peripheral whole blood.

When the blood used in this step is frozen or refrigerated blood, the blood can be thawed or heated for use by a method known in the art. Examples thereof include a method involving adding a RPMI-1640 medium, for thawing, to the PBMCs cryopreserved, and then incubating the thawed PBMCs at 37° C. for 3 hours under 5% CO² condition.

(3) NK Cell Growth-Stimulating Factor

In the present invention, the “NK cell growth-stimulating factor” refers to a factor directly or indirectly enriching NK cells. Examples of the directly enriching factor include factors having the function of transmitting growth signals or activation signals into NK cells through the specific binding to the surface receptors of the NK cells. Examples of the indirectly inducing factor include factors inducing the production and release of liquid factors such as cytokines through the binding to the surface receptors of cells other than NK cells, such as monocytes. In this case, the NK cells are indirectly enriched by the released liquid factor.

The NK cell growth-stimulating factors of the present invention comprise an anti-CD16 antibody, an anti-CD137 antibody, OK432, and a cytokine as essential factors.

The “anti-CD16 antibody” refers to an antibody against an antigen CD16. The antigen CD16 serves as a marker for NK cells or granulocytes and is known as a protein FcγRIII constituting Fc receptor present on the surfaces of most of NK cells in the resting period. The NK cell growth-inducing activity of the anti-CD16 antibody was found by JP Patent No. 4275680 and had been unknown before then. Although the mechanism underlying the induction of NK cell growth by the anti-CD16 antibody remains to be elucidated, the co-addition of the anti-CD16 antibody and a cytokine such as IL-2 can drastically increase the induction rate of NK cell growth compared with the addition of the cytokine alone (JP Patent No. 4275680; and Non Patent Literature 1). This antibody can be any of monoclonal and polyclonal antibodies and fragments thereof.

In the present specification, the “fragments thereof” are partial fragments of a polyclonal or monoclonal antibody and refer to polypeptide chains or complexes thereof having activity substantially equivalent to the antigen-specific binding activity of the antibody. The fragments thereof correspond to, for example, antibody portions containing at least one antigen binding site, i.e., polypeptide chains having at least one set of a light chain variable region (VL) and a heavy chain variable region (VH), or complexes thereof. Specific examples thereof include a large number of sufficiently characterized antibody fragments formed by the cleavage of immunoglobulins with various peptidases. These antibody fragments correspond to, for example, Fab, F(ab′)₂ and Fab′. Any of these antibody fragments contain the antigen binding site and have the ability to specifically binding to the antigen (i.e., here, CD16).

In the present specification, the monoclonal antibody may be a synthetic antibody synthesized chemically or by use of a recombinant DNA method. Examples thereof include antibodies constructed by use of a recombinant DNA method. Specifically, the synthetic antibody corresponds to, but is not limited to, a monomeric polypeptide molecule comprising one or more VLs and one or more VHs of the monoclonal antibody of the present invention artificially linked via a linker peptide or the like having an appropriate length and sequence, or a multimeric polypeptide (multivalent antibody) thereof. Examples of such a polypeptide include single chain fragment of variable region (scFv), diabody, triabody and tetrabody. The antigen binding sites of a divalent or higher multivalent antibody such as diabody do not have to bind to the same epitope and may have multispecificity that allows these antigen binding sites to respectively recognize and specifically bind to different epitopes. The antibody preferable as the anti-CD16 antibody of the present invention is a monoclonal antibody, i.e., an anti-CD16 monoclonal antibody. An anti-human CD16 monoclonal antibody against human CD16 as an antigen is particularly preferable. A commercially available product can also be used as such an antibody. Examples thereof include anti-human CD16 monoclonal antibodies 3G8 and B73.1.

The “anti-CD137 antibody” refers to an antibody against an antigen CD137. The antigen CD137 is a 30 kDa glycoprotein belonging to the costimulatory molecule TNF receptor superfamily. Activation by the anti-CD137 antibody has been shown to contribute to the activation of T cells and the maintenance of activated T cells and memory T cells (Schwarz H, et al. 1996, Blood 87: 2839-2845; and Croft M, et al. 2003; Nat Rev Immulo. 3: 609-620). On the other hand, none of previously known reports have demonstrated, for example, the activation of human NK cells by this antibody (Baessler T, et al. 2010; Blood 115: 3058-3069). The anti-CD137 antibody used in the NK cell growth-stimulating factors of the present invention is not particularly limited as long as the antibody specifically recognizes and binds to the antigen CD137. The anti-CD137 antibody may include monoclonal and polyclonal antibodies and fragments thereof. A monoclonal antibody is preferable. A commercially available antibody can also be used as the monoclonal antibody of the present invention. Examples thereof include anti-human CD137 monoclonal antibodies 4-1BB, G6, 4B4-1, O.N.185, BBK-2, C-20, D-20, G-1, N-16, BBEX2 and Lq-14.

The “OK432” (trade name: Picibanil) refers to an antitumor agent comprising a penicillin-treated Su strain of hemolytic streptococcus (type III group A Streptococcus pyogenes) as an active ingredient and belongs to the BRM described above. The “BRM” refers to, as described above, a substance that brings about therapeutic effects by modifying the biological response of hosts to tumor cells as described above. OK432 is known to serve as an immune adjuvant capable of activating, for example, monocytes, through the binding to the surface TLR of the monocytes so that immune response is activated (Ryoma Y, et al., 2004, Anticancer Res., 24: 3295-301.).

The “cytokine” refers to a wide variety of proteinous hormones that play a role in signal transduction between cells, and has the effect of enriching lymphocytes such as T cells or NK cells as described above in the immune system. Examples thereof include interleukin, interferon (INF), TNF and MCP. Examples of the cytokine preferable for the NK cell growth-stimulating factors of the present invention include interleukin 2 (hereinafter, referred to as “IL-2”; the same holds true for other interleukins), IL-12, IL-15, IL-18, TNF-α and IL-1β. Of them, IL-2 is a particularly preferable cytokine in the present invention.

The NK cell growth-stimulating factors of the present invention can optionally further comprise, in addition to the essential factors described above, an anti-CD3 antibody, a bisphosphonate derivative or a salt thereof, or a hydrate thereof (hereinafter, referred to as a “bisphosphonate derivative, etc.”), and/or BRM other than OK432, etc.

The “anti-CD3 antibody” refers to an antibody against CD3. The anti-CD3 antibody used as an NK cell growth-stimulating factor of the present invention is not particularly limited as long as the antibody specifically recognizes CD3 and binds thereto. This antibody can be any of monoclonal and polyclonal antibodies. A monoclonal antibody is preferable. Examples thereof include muromonab-CD3 (trade name: Orthoclone OKT3 (registered trademark), Janssen Pharmaceutical K.K.).

The “bisphosphonate derivative” refers to a compound represented by the following general formula 1:

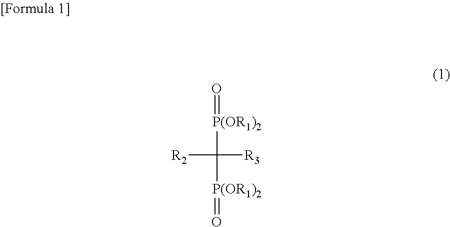

In the formula, R₁ represents a hydrogen atom (H) or a lower alkyl group; and R₂ and R₃ each independently represent a hydrogen atom, halogen, a hydroxyl group, an amino group, a thiol group, a substituted or unsubstituted aryl group, a substituted or unsubstituted alkyl group, a lower alkylamino group, an aralkyl group, a cycloalkyl group, or a heterocyclic group, or R₂ and R₃ form a portion of a cyclic structure containing them wherein substituents forming the cyclic structure are each independently derived from halogen, a lower alkyl group, a hydroxyl group, a thiol group, an amino group, an alkoxy group, an aryl group, an arylthio group, an aryloxy group, an alkylthio group, a cycloalkyl group, or a heterocyclic group in R₂ and R₃.

Specific examples of the bisphosphonate derivative include zoledronic acid, pamidronic acid, alendronic acid, risedronic acid, ibandronic acid, incadronic acid, and etidronic acid.

In the present invention, one or more bisphosphonate derivatives or the like can be added as the NK cell growth-stimulating factor. In the present invention, the bisphosphonate derivative is particularly preferably zoledronic acid or a zoledronic acid derivative capable of inducing the enrichment activation of NK cells or a salt thereof, or a hydrate thereof.

The zoledronic acid (trade name: Zometa (registered trademark), Novartis Pharma K.K.) is bisphosphonate having bone resorption inhibitory activity and is known as a therapeutic drug for hypercalcemia caused by malignant tumor, bone lesions attributed to multiple myeloma, and bone lesions attributed to solid cancer metastasized to bone. Since its chemical structure incorporates nitrogen-containing bisphosphonates (N—BPs), this acid inhibits the intracellular synthesis of farnesyl pyrophosphate (FPP), resulting in the accumulation of its precursor isopentenyl pyrophosphate (IPP). Thereby, the immune response of the organism can be activated (van Beek E, et al., 1999, Biochem Biophys Res Commun, 264: 108-11; and Gober H J, et al., 2003, J Exp Med, 197: 163-8.), and γδT cell growth activity can be enhanced (Sato K, et al., 2005, Int. J. Cancer, 116: 94-99; and Kondo M, et al., 2008, Cytotherapy, 10 (8): 842-856.).

The “salt thereof” refers to a base-addition salt of the bisphosphonate derivative, preferably zoledronic acid. Examples of the base-addition salt include: alkali metal salts such as sodium salt and potassium salt; alkaline earth metal salts such as calcium salt and magnesium salt; aliphatic amine salts such as trimethylamine salt, triethylamine salt, dicyclohexylamine salt, ethanolamine salt, diethanolamine salt, triethanolamine salt, and procaine salt; aralkylamine salts such as N,N-dibenzylethylenediamine; heterocyclic aromatic amine salts such as pyridine salt, picoline salt, quinoline salt, and isoquinoline salt; basic amino acid salts such as arginine salt and lysine salt; and ammonium salt and quaternary ammonium salts such as tetramethylammonium salt, tetraethylammonium salt, benzyltrimethylammonium salt, benzyltriethylammonium salt, benzyltributylammonium salt, methyltrioctylammonium salt, and tetrabutylammonium salt.

Examples of “BRM other than OK432” include protein-polysaccharide complexes extracted from basidiomycetes, more specifically, lentinan extracted from Lentinula edodes and Krestin (registered trademark) extracted from Trametes versicolor.

(4) Stimulation Method

The term “stimulating” refers to contacting the NK cell growth-stimulating factors with NK cells to thereby induce the enrichment of the NK cells.

In a specific stimulation method thereto, first, blood, for example, PBMCs, collected from an organism is adjusted with a medium into, for example, a cell density of 1×10⁶ to 3×10⁶ cells/mL. In this context, the medium used may be any appropriate medium for cell culture supplemented with inactivated human serum or plasma at a volume ratio (V/V) on the order of 5 to 10%. When the blood preparation is predicated on administration for the adoptive immunotherapy, desirably, a serum-free medium for adoptive immunotherapy such as OpTmizer supplemented with autologous plasma is used as the medium. The autologous plasma can be prepared from blood obtained after the blood collection step, as described above. For example, the collected peripheral whole blood is centrifuged at 3000 rpm at room temperature (10° C. to 30° C.: the same holds true for the description below) for approximately 10 minutes to obtain a supernatant, which can in turn be used as the autologous plasma. If necessary, the medium may be further supplemented with an antibiotic such as streptomycin, penicillin, kanamycin, or gentamicin.

Next, each NK cell growth-stimulating factor is added to the culture solution containing the preliminarily prepared PBMCs.

For stimulation with the anti-CD16 antibody, this antibody can be added directly to the medium at, for example, 0.01 μg/mL to 100 μg/mL, preferably 0.1 μg/mL to 10 μg/mL, more preferably 1 μg/mL, in terms of the final concentration, or can be added thereto in a form immobilized on a solid-phase support. The addition of the antibody in a form immobilized on a solid-phase support is preferable. This is because the anti-CD16 antibody thus immobilized on a solid phase can come into contact with NK cells with increased frequency in the constant direction and thus efficiently impart growth stimulation to the NK cells compared with a free form. In this context, the “support” refers to a scaffold for antibody immobilization. A material for the support is not particularly limited as long as this material permits stable immobilization of the antibody. For example, a synthetic resin (e.g., plastic), glass, or a metal can be used. The shape of the support is not particularly limited. A shape with a large surface area of contact with the culture solution is preferable because the antibody immobilized on this support can come into contact with the NK cells with higher frequency. Examples thereof include spherical beads and porous cubes having lymphocyte-sized pores.

When the support is made of a material with high affinity for the antibody, for example, plastic, the anti-CD16 antibody can be immobilized on this support by a simple method of contacting (including dipping, coating, circulating, spraying, etc.) the antibody solution with the support and keeping them at a predetermined temperature for a predetermined time. The anti-CD16 antibody solution can be obtained, for example, by dissolving the anti-CD16 antibody in sterile distilled water or a medium for cell culture, then sterilizing, if necessary, by filtration through, for example, a filter of 0.22 μm in pore size, and adjusting the filtrate with sterile distilled water or a medium to 1 μg/mL in terms of the final concentration. For immobilizing the anti-CD16 antibody on the support, it is preferred to use the anti-CD16 antibody solution in a volume in consideration of the surface area or the like of the solid-phase support. For example, approximately 15 mL of 1 μg/mL anti-CD16 antibody solution can be used for immobilization on a plastic flask having an inner wall surface area of 150 cm². Alternatively, 5 mL and 10 mL of the solution can be used for culture flasks having inner wall surface areas of 25 cm² and 75 cm², respectively. The anti-CD16 antibody is attached to the support by subsequent incubation at 37° C. for 12 to 24 hours. Alternatively, a commercially available antibody immobilization kit or the like may be used. For example, CarboLink (Pierce Biotechnology, Inc.) can be used. Such an immobilization kit is useful for supports made of a material difficult to attach to antibodies.

After the immobilization of the anti-CD16 antibody on the support, desirably, the support with the anti-CD16 antibody immobilized thereon is washed, if necessary, to remove the anti-CD16 antibody solution. For example, the support can be washed several times, for example, approximately 2 to 5 times, with an appropriate amount of PBS. Such a culture container comprising the anti-CD16 antibody thus immobilized on the support can be stored at 0° C. to 8° C., preferably 3° C. to 6° C., and thereby used for approximately 1 month without reducing or inactivating the avidity of the antibody.

For stimulation with the anti-CD137 antibody, an OK432 solution can be added at, for example, 0.1 μg/mL to 10 μg/mL, in terms of the final concentration to the culture solution containing PBMCs. This is because: a final concentration lower than 0.1 μg/mL is insufficient for inducing growth stimulation; and a final concentration higher than 10 μg/mL rather inhibits the growth of NK cells. This final concentration is preferably 0.3 μg/mL to 6 μg/mL, more preferably 1 μg/mL to 3 μg/mL.

For stimulation with OK432, an OK432 solution can be added at, for example, 0.005 KE/mL to 0.05 KE/mL, preferably 0.008 KE/mL to 0.015 KE/mL, more preferably 0.01 KE/mL, in terms of the final concentration to the culture solution containing PBMCs. The OK432 solution can be prepared by dissolving Picibanil (5 KE/vial; Chugai Pharmaceutical Co., Ltd.) in 2 mL of water (e.g., injectable water).

For stimulation with the cytokine, one type of cytokine may be added thereto, or a combination of several types of cytokines may be added thereto. In consideration of cost, etc., it is preferred to add only IL-2. The amount of, for example, IL-2, added is preferably in the range of 100 units (U)/mL to 2000 U/mL, in terms of the final concentration. This is because: an amount smaller than 100 U/mL is insufficient for inducing growth stimulation; and an amount larger than 2000 U/mL does not offer the growth of NK cells according to increase in IL-2 concentration. This amount is preferably in the range of 700 U/mL to 2000 U/mL.

When the NK cell growth-stimulating factors further comprise an anti-CD3 antibody and/or a bisphosphonate derivative, etc., the anti-CD3 antibody can be added at, for example, 0.01 ng/mL to 1000 ng/mL, preferably 0.1 ng/mL to 10 ng/mL, more preferably 1 ng/mL, in terms of the final concentration to the culture solution containing PBMCs. When the NK cell growth-stimulating factors comprise a bisphosphonate derivative, etc., 4 mg/vial of a zoledronic acid hydrate injection (2.94 μmol/mL; Novartis Pharma K.K.) can be used directly. Alternatively, zoledronic acid can be added to the medium at, for example, 1 μM/mL to 10 μM/mL, preferably 3 μM/mL to 7 μM/mL, more preferably 5 μM/mL, in terms of the final concentration.

For sufficiently stimulating PBMCs, it is preferred to keep the blood at a physiological cell temperature described later for 1 to 3 days after the addition of each of these NK cell growth-stimulating factors. This period may be promoted concurrently with the period of the subsequent culture step. The NK cells, etc. can be cultured with stimulation applied thereto.

During the period for which the blood is kept at a physiological cell temperature, high-temperature stimulation may be applied thereto at 38° C. to 40° C. for a period of 10 hours to 30 hours. This high-temperature stimulation can further activate the NK cells. A temperature lower than 37° C. at which the blood is kept in the stimulation step is not preferable because the temperature fails to sufficiently activate lymphocytes. A temperature higher than 40° C. is not preferable because the temperature makes lymphocytes more likely to be degenerated or damaged by heat.

Means of keeping the blood at a predetermined temperature is not particularly limited as long as the blood can be kept at the constant temperature by this means. Examples thereof include means by which the blood together with its container is set at a predetermined temperature using a CO₂ incubator.

1-2-2. Culture Step

The “culture step” is the step of culturing the blood at a physiological cell temperature after the stimulation step. The feature of this step is that the number of the NK cells is increased with their enrichment maintained.

The “physiological cell temperature” refers to the optimum temperature for culturing the cell. The physiological cell temperature is usually the body temperature of a mammal that has provided the blood used. Thus, when the mammal is a human, this temperature is generally 37° C. and may be the temperature with a tolerance of less than ±0.5° C., i.e., 36.5 to 37.5° C. This is because the internal temperature of an incubator might fluctuate within this temperature range.

At the initial stage of this step, the period for which the NK cells are sufficiently stimulated with the NK cell growth-stimulating factors added in the stimulation step can be secured and also be concurrent with the period for which these cells are cultured. After sufficient stimulation, it is preferred to temporarily remove the NK cell growth-stimulating factors from the medium to thereby cancel the stimulation step. This is because, although most of factors such as cytokines can continue to impart enrichment-inducting stimulation to the NK cells even in the culture step, the long-term stimulation of the NK cells with the anti-CD16 antibody, the anti-CD137 antibody, OK432, the anti-CD3 antibody, and/or zoledronic acid or the like might have undesired influence, for example, apoptosis, on NK cell enrichment. These stimulating factors can be removed by a method involving, for example, collecting PBMCs from the culture solution after the stimulation step and then transferring these PBMCs to a new culture solution free from the anti-CD16 antibody, the anti-CD137 antibody and OK432, the optional anti-CD3 antibody, optional zoledronic acid or the like. The removal of the factors and the collection of PBMCs are achieved by centrifuging the culture solution that has undergone the stimulation step and removing the supernatant. Its specific method can follow a medium replacement method described below.

The culture is performed for 7 days to 30 days, preferably 9 days to 28 days, 12 days to 26 days, or 14 days to 24 days, in a 5% CO₂ incubator that satisfies the physiological cell temperature condition.

For the culture period, it is preferred to add a fresh medium or replace the medium by a fresh medium at regular intervals of 2 days to 5 days. As a specific example of the medium replacement, first, the culture solution containing NK cells after the stimulation step is transferred to an already sterilized centrifuge tube. Subsequently, the tube is centrifuged at approximately 1200 rpm at room temperature for approximately 8 minutes, and the supernatant is then removed, or the precipitates containing NK cells are collected. The collected cell precipitates are transferred at a cell density of 0.6 to 1.0×10⁶ cells/mL to a fresh culture solution containing IL-2 and plasma. In this context, the cytokine such as IL-2 can be added thereto at approximately 300 U/mL to approximately 700 U/mL in terms of the final concentration. This is because the NK cells have already been activated after the stimulation step and produce cytokines such as IL-2 in themselves.

The medium used in the culture can be any general medium for use in cell culture as a rule. Examples thereof include AIM-V medium (Life Technologies Corp.), RPMI-1640 medium (Life Technologies Corp.), Dulbecco's modified eagle's medium (DMEM; Life Technologies Corp.), OpTmizer T-cell Expansion SFM (Life Technologies Corp.), TIL (Immuno-Biological Laboratories Co, Ltd.), epidermal keratinocyte medium (KBM; Kohjin Bio Co., Ltd.), Iscove's medium (IMEM; Life Technologies Corp.) and Alys medium (Cell Science & Technology Institute, Inc.). OpTmizer medium is preferable.

After the culture, the culture solution is confirmed to be free from contamination with bacteria or endotoxin. The presence or absence of bacteria can be examined by colony formation assay, while the presence or absence of endotoxin can be examined by colorimetry such as commercially available ELISA or by a suspension method such as limulus test.

1-3. Effect

The method for producing an NK cell-enriched blood preparation according to this aspect can produce an NK cell-enriched blood preparation from blood collected from an organism.

According to the method for producing an NK cell-enriched blood preparation according to this aspect, the essential step of keeping blood at a predetermined temperature for a predetermined time (activation step; which corresponds to the optional high-temperature stimulation in the stimulation step of the present invention) in JP Patent No. 4275680 is no longer essential. As a result, an incubator set to a predetermined temperature necessary for the activation step is not necessarily required. This can drastically reduce burdens from an equipment standpoint in research facilities where the present invention is carried out, or burdens in terms of operation/management by an operator.

Moreover, the production method can minimize physical burdens on donors because the blood collected from an organism may be peripheral blood.

The production method does not require particular special equipment or the like and can utilize regular equipment or the like installed in general testing facilities, research facilities, etc., for cell culture. In addition, any of necessary reagents, etc. can be obtained easily. Accordingly, the production method of this aspect can be carried out advantageously in research facilities capable of aseptic manipulation, such as clean room, substantially without the need of initial equipment investment or the like.

The NK cell-enriched blood preparation obtained by the production method of this aspect is capable of preventing the recurrence of cancer or effectively treating advanced cancer in actual clinical experiments. In addition, the blood preparation that can be provided is safe in such a way that the administration of the blood preparation has been confirmed to have no adverse reaction.

When the bisphosphonate derivative is used as one of the NK cell growth-stimulating factors, the bisphosphonate derivative has the effect of enhancing γδf cell growth activity. As a result, a remarkable γδT cell growth effect can be obtained, in addition to the growth of NK cells.

### 2. NK Cell-Enriched Blood Preparation

2-1. Summary

The second aspect of the present invention relates to an NK cell-enriched blood preparation obtained by the production method of the first aspect.

2-2. Constitution

The NK cell-enriched blood preparation of this aspect can be obtained from the culture solution that has undergone the culture step in the first aspect. However, the NK cell-enriched blood preparation does not require the medium used in the culture or the growth-stimulating factors added to the medium. Thus, for use of the NK cell-enriched blood preparation, it is preferred to remove the medium and the growth-stimulating factors as much as possible from the culture solution to adjust enriched NK cells, etc. As a specific example, the medium and the growth-stimulating factors are removed by a method involving first transferring the culture solution containing the grown/activated NK cells to an already sterilized centrifuge tube, which is then centrifuged at 1200 rpm at room temperature for approximately 8 minutes to remove the medium in a supernatant containing the growth-stimulating factors. The NK cells can be collected as precipitates. It is preferred to wash the collected NK cells two or more times with PBS (−). The number of the NK cells thus washed is counted using a hemacytometer and adjusted with 10 mL to 200 mL of a lactate Ringer solution or saline. In this way, the NK cell-enriched blood preparation of this embodiment can be adjusted. If necessary, cytokines or the like may be added to the blood preparation.

For obtaining sufficient effects using the blood preparation of this embodiment, it is preferred that 70% or more of the number of the NK cells contained therein should be in an activated state. The activation of the NK cells can be determined by examining cytotoxic activity against a leukemia cell line K562 or activation marker expression. A marker known in the art, such as CD69, can be used as the activation marker. An antibody against each marker can be used in the detection thereof.

The NK cell-enriched blood preparation of this embodiment may be used immediately after its production or may be stored either for a predetermined period at a temperature of 0° C. to 8° C. or for a period as long as several years at a ultralow temperature (approximately −80° C.) or in liquid nitrogen after being supplemented with a storage solution or the like. A commercially available lymphocyte storage solution can be used conveniently as the storage solution. For example, Bambanker (Nippon Genetics Co., Ltd.) or KM Banker II (Cosmo Bio Co., Ltd.) can be used.

2-3. Effect

Since the NK cell-enriched blood preparation of this aspect contains 10×10⁹ to 100×10⁹ NK cells from 20 mL to 60 mL of peripheral whole blood, the number of NK cells in a test subject can be rapidly increased by the administration of the blood preparation. Thus, the natural immune system of a test subject having disease such as tumor can be enhanced by the administration of the NK cell-enriched blood preparation. As a result, the progression of the disease can be delayed, or the disease can be cured.

According to the NK cell-enriched blood preparation of this aspect, a blood preparation containing a large number of enriched NK cells can be cryopreserved and can therefore be administered in a necessary amount to a test subject at the time of need.

### 3. Composition for NK Cell Enrichment

3-1. Summary

The third aspect of the present invention relates to a composition for NK cell enrichment. The composition for NK cell enrichment of this aspect can be added to blood, preferably a medium containing PBMCs, to thereby conveniently and efficiently enriching NK cells in the medium.

3-2. Constitution

The “composition for NK cell enrichment” refers to a composition capable of enriching NK cells present in a medium through its addition to the medium.

The composition for NK cell enrichment of this aspect comprises the anti-CD16 antibody, the anti-CD137 antibody, the OK432, and the cytokine described in the first embodiment and optionally further comprises the anti-CD3 antibody and/or the bisphosphonate derivative, etc., represented by the formula 1. The anti-CD16 antibody is preferably an anti-human CD16 monoclonal antibody such as 3G8. The anti-CD137 antibody is preferably an anti-human CD137 monoclonal antibody such as 4-1BB. The cytokine is preferably a compound selected from the group consisting of IL-2, IL-12, IL-15, TNF-α, IL-1β, and IL-18, more preferably IL-2. Also, the anti-CD3 antibody is preferably an anti-human CD3 monoclonal antibody such as muromonab-CD3. The bisphosphonate derivative is preferably a compound selected from the group consisting of zoledronic acid, pamidronic acid, alendronic acid, risedronic acid, ibandronic acid, incadronic acid, and etidronic acid, more preferably, zoledronic acid. The composition for NK cell enrichment may additionally incorporate medium components for lymphocytes, such as RPMI-1640, a pH stabilizer, an antibiotic, etc.

These components for the composition can be mixed in amounts that give their respective predetermined final concentrations when added to a predetermined amount of a medium. Specifically, these components can be mixed so that: the anti-CD16 antibody gives a final concentration of 0.01 μg/mL to 100 μg/mL, preferably 0.1 μg/mL to 10 μg/mL, more preferably 1 μg/mL; OK432 gives a final concentration of 0.005 KE/mL to 0.05 KE/mL, preferably 0.008 KE/mL to 0.015 KE/mL, more preferably 0.01 KE/mL; the anti-CD137 antibody gives a final concentration of 0.1 μg/mL to 10 μg/mL, preferably 0.3 μg/mL to 6 mg/mL, more preferably 1 μg/mL to 3 μg/mL; and the cytokine (preferably, IL-2) gives a final concentration of 200 U/mL to 2000 U/mL, preferably 700 U/mL to 1500 U/mL, more preferably 1000 U/mL. When the composition further comprises an anti-CD3 antibody and/or a bisphosphonate derivative or the like, the anti-CD3 antibody (preferably, muromonab-CD3) can be mixed therewith so that the component gives a final concentration of 0.01 ng/mL to 1000 ng/mL, preferably 0.1 ng/mL to 10 ng/mL, more preferably 1 ng/mL, and when the composition further comprises a bisphosphonate derivative or the like (preferably, zoledronic acid), this component can be mixed therewith at a final concentration of 1 μM/mL to 10 μM/mL, preferably 3 μM/mL to 7 μM/mL, more preferably 5 μM/mL.

The dosage form of the composition is not particularly limited. The composition can be in a liquid form dissolved in an appropriate buffer, in a powdery form, or in the form of tablets prepared from a powder supplemented with an appropriate excipient, etc. Alternatively, the composition may be a mixture of different forms. For example, the composition is in a dosage form in which the anti-CD16 antibody immobilized on a solid-phase support such as plastic beads is mixed with a solution containing the OK432, the anti-CD137 antibody, and the cytokine, and optionally, the anti-CD3 antibody and/or the bisphosphonate derivative or the like represented by the formula 1.

3-3. Effect

According to the composition for NK cell enrichment of this aspect, NK cells can be enriched by simple procedures of addition to a predetermined amount of an appropriate cell culture solution containing the NK cells and subsequent culture.

### 4. Kit for Production of NK Cell-Enriched Blood

4-1. Summary

The fourth aspect of the present invention relates to a kit for production of NK cell-enriched blood. The kit of this aspect can be used in the culture of blood, preferably PBMCs, to thereby conveniently and easily produce an NK cell-enriched blood preparation.

4-2. Constitution

The kit for production of NK cell-enriched blood of this aspect comprises the anti-CD16 antibody, the anti-CD137 antibody, the OK432, and the cytokine described in the first embodiment and optionally comprises the anti-CD3 antibody, the bisphosphonate derivative represented by the formula 1, and/or BRM other than OK432, etc. The kit for production of NK cell-enriched blood may additionally incorporate sterile water or a buffer for dissolving each NK cell growth-stimulating factor, an instruction manual, etc.

The anti-CD16 antibody and the anti-CD137 antibody incorporated in this kit and the anti-CD3 antibody optionally added thereto can be antibodies capable of specifically recognizing the antigen CD16, the antigen CD137, and the antigen CD3, respectively, and binding thereto and may each be a monoclonal antibody or a polyclonal antibody. A monoclonal antibody is preferable. The anti-CD16 antibody is more preferably immobilized on an appropriate solid-phase support. Specific examples of the anti-CD137 antibody include 4-1BB. The cytokine is preferably a compound selected from the group consisting of IL-2, IL-12, IL-15, TNF-α, IL-1β and IL-18, more preferably IL-2.

Specific examples of the anti-CD3 antibody incorporated in this kit include muromonab-CD3 (trade name: Orthoclone OKT3 (registered trademark), Janssen Pharmaceutical K.K.). The bisphosphonate derivative is preferably a compound selected from the group consisting of zoledronic acid, pamidronic acid, alendronic acid, risedronic acid, ibandronic acid, incadronic acid, and etidronic acid, more preferably zoledronic acid.

These NK cell growth-stimulating factors can be incorporated alone or in combination of two or more thereof in the kit. For example, the NK cell growth-stimulating factors other than the anti-CD16 antibody may be packaged each individually and incorporated in the kit, or some or all of them may be incorporated in one portion in the kit. The state of each NK cell growth-stimulating factor is not particularly limited. One NK cell growth-stimulating factor may be in a liquid state while the other NK cell growth-stimulating factors may be in a solid state. Particularly, it is preferred that the anti-CD16 antibody should be incorporated therein in a form immobilized on an appropriate solid-phase support such as plastic beads.

### 5. Cellular Immunotherapy to Treat Disease

5-1. Summary

The fifth aspect of the present invention relates to cellular immunotherapy for treating a disease, involving administering the NK cell-enriched blood preparation produced in the first aspect to an organism to enhance its immunity.

5-2. Constitution

This aspect relates to cellular immunotherapy involving administering the NK cell-enriched blood preparation obtained by the production method of the first aspect to an organism.

The “cellular immunotherapy” according to this aspect refers to a method for treating a disease, involving administering the NK cell-enriched blood preparation obtained by the production method of the first aspect to an organism to enhance the immunity of the organism. Particularly, for the cellular immunotherapy of this aspect, it is preferred to be predicated on adoptive immunotherapy. This is because the adoptive immunotherapy is substantially free from the risk of rejection, as described above.

The NK cell-enriched blood preparation to be administered contains a larger number of activated NK cells having immunity against cancer, viral infection, bacterial infection, or parasitic infection than the average number thereof in usual blood per unit volume. In this context, the “cancer” means general malignant tumor. The cancer corresponds to, for example, epithelial tumor, sarcoma, leukemia, and myeloma. Specific examples thereof include brain tumor, retinoblastoma, basal cell cancer, malignant melanoma, tongue cancer, esophageal cancer, stomach cancer, colon cancer, lung cancer, leukemia, lymphoma, breast cancer, uterine cervical cancer, uterine body cancer, ovary cancer, prostate cancer, testis tumor, bladder cancer, kidney cancer, liver cancer, pancreas cancer, and fibrosarcoma. In this context, the “viral infection” refers to general disease caused by infection with a virus and particularly corresponds to the intractable chronic viral infection and acute viral infection. Examples of the intractable chronic viral infection include HIV infection causative of AIDS, chronic viral hepatitis, and human papillomavirus infection causative of uterine cervix cancer. Examples of the acute viral infection include viral respiratory infection such as influenza, and acute viral infection in an immunodeficient state. The “bacterial infection” refers to disease caused by infection with an eubacterium (including Gram-positive bacteria and Gram-negative bacteria) or a fungus (including filamentous bacteria, yeast, or the like, and basidiomycetes). Examples thereof include candidal infection, blastomycosis, and histoplasmosis. In this context, the “parasitic infection” refers to general disease caused by protozoan or helminth. Examples thereof include malaria, leishmaniasis, filaria, echinococcosis, and schistosomiasis japonicum.

The “lymphocyte having immunity” means a lymphocyte having fortified functions in the immune system. Such lymphocytes correspond to, for example, NK cells, killer T cells, γδT cells, and NKT cells that have been activated to be cytotoxic. In this context, the “average value in usual blood per unit volume” means the average number per unit volume of blood cells having immunity against cancer, viral infection, or fungal infection generally observed in the blood of a healthy individual. For example, approximately 5×10⁵ NK cells on average are present per mL of blood of a healthy adult individual.

5-3. Method

Hereinafter, a method for administering the NK cell-enriched blood preparation in the cellular immunotherapy of this aspect will be described by taking adoptive immunotherapy as an example. The administration method is basically the same as a known method performed in the conventional adoptive immunotherapy except that the NK cell-enriched blood preparation of the first aspect is administered. Thus, the administration method can be performed according to that of the adoptive immunotherapy known in the art. Examples thereof include methods involving administering the blood preparation produced by the method for producing an NK cell-enriched blood preparation according to the first aspect from blood collected from a patient, into the body of the patient using, for example, intravenous injection or drip infusion approximately 2 weeks later.

One dose of the NK cell-enriched blood preparation according to this aspect can be a volume containing NK cells in the range of 20×10⁷ to 5×10⁹ cells for a human. This dose is intended for a general adult. For actual administration, it is preferred to appropriately adjust the dose in consideration of the age, sex, body weight, disease conditions, body strength, etc., of a recipient of the blood preparation.

One example of the cellular immunotherapy of this aspect includes 1 course (6 cycles) or longer of continuous administration at approximately 2-week intervals with the above-described administration method defined as one cycle. Cellular immunotherapy other than adoptive immunotherapy can also be performed in the same way as above except that an NK cell-enriched blood preparation obtained from a non-self organism is administered.

5-4. Effect

The cellular immunotherapy of this aspect has high efficacy on the healing of a disease such as cancer, compared with many conventional immunotherapy methods, particularly, adoptive immunotherapy. A person skilled in the conventional adoptive immunotherapy can carry out the cellular immunotherapy of this embodiment without acquiring particular skills because this cellular immunotherapy can be operated by the same basic technique, etc., as in the conventional adoptive immunotherapy.

### EXAMPLES

Hereinafter, the present invention will be described specifically with reference to Examples. Examples below are provided merely for illustrative purposes of the present invention, and the present invention is not intended to be limited to these Examples by any means. In this context, small experimental errors and deviations are tolerated for numeric values as to temperature, amount, time, etc., used in these Examples.

### Example 1

### Method for Producing NK Cell-Enriched Blood Preparation (1)

The first aspect of the present invention will be described with reference to a specific example of the method for producing the blood preparation used in adoptive immunotherapy. In Examples 1 to 3 of the present specification, healthy individuals were used as donors instead of actual individuals to be treated such as cancer patients.

(1) Preparation of Autologous Plasma

First, autologous plasma for cell culture was prepared. 40 mL of peripheral whole blood was collected from the vein of each donor into a blood collection tube supplemented with 50 U/mL heparin. The collected peripheral whole blood was transferred to a sterile conical centrifuge tube and centrifuged at 3000 rpm for 10 minutes. Then, the supernatant was separated as plasma. To the remaining blood cell components after the plasma collection, sterile PBS (−) was added in an amount 3 times that of the whole blood before plasma separation to prepare a “blood cell component solution”, which was in turn used in the subsequent preparation of PBMCs. The plasma was inactivated by treatment at 56° C. for 30 minutes and further centrifuged at 3000 rpm for 10 minutes to remove platelet, etc. Then, the plasma was stored at 4° C. This plasma was intended as autologous plasma for cell culture to be added to a medium, and used in a necessary amount every time a medium was prepared.

(2) Preparation of PBMCs

A specific gravity solution was layered onto the blood cell component solution. Erythrocytes or granulocytes were removed using a density-gradient centrifugation method to isolate PBMCs. The specific gravity solution used was Ficoll-Paque PLUS (GE Healthcare (formerly Amersham Biosciences Corp.)), and the operational procedures followed the protocol supplied with the kit. The collected PBMCs were washed 2 or 3 times by the addition of 30 mL of serum-free PBS (−). After the washing, an aliquot was sampled from the obtained suspension of PBMCs and stained with a Turk's solution, and the number thereof was then counted using a hemacytometer. As a result, 3.4×10⁷ PBMCs were collected from 40 mL of peripheral whole blood. The PBMCs thus collected were added and suspended at a cell density of 1×10⁶ cells/mL to OpTmizer (Life technologies Corp.) medium supplemented with 5% (V/V) of the autologous plasma.

(3) Stimulation Step

0.2 mg of an anti-human CD16 antibody (Clone 3 GB, Beckman Coulter, Inc.) was dissolved in 1 mL of sterile distilled water. Since this anti-CD16 antibody is not a sterile product, this solution was sterilized by filtration through a 0.22 μm filter. The solution was adjusted to 1 μg/mL in terms of the final concentration by the addition of 199 mL of sterile distilled water, and then mixed. After filtration, 5 mL of the anti-CD16 antibody solution was placed in a 25 cm² culture flask and left standing overnight at 37° C. to immobilize the anti-CD16 antibody in this solution onto the inner wall of the flask. Then, the solution was discarded, and the inside of the flask was washed twice with sterile PBS (−).

5 mL of the prepared suspension of PBMCs was transferred into the flask. Subsequently, 25 μL of 0.2 μg/μL solution of an anti-human CD137 antibody (4-1BB, BioLegend, Inc.), 20 μL of 4 μL/mL (final concentration) aqueous OK432 (Picibanil; Chugai Pharmaceutical Co., Ltd.) solution, and 4 μL of 900 U/μL IL-2 (Proleukin; Chiron Corp.) solution were added to the suspension of PBMCs, and the mixture was sufficiently stirred.

In order to sufficiently stimulate PBMCs with each of the NK cell growth-stimulating factors, the culture flask was transferred to a 5% CO₂ incubator preset to a chamber temperature of 37° C., and kept for 3 days.

(4) Culture Step

In order to remove the NK cell growth-stimulating factors from the culture solution, 5 mL of the culture solution was then collected into a conical centrifuge tube and centrifuged at 1200 rpm for 8 minutes. After the centrifugation, the medium in the supernatant was removed, and the cell pellet was suspended in 4 mL of OpTmizer medium containing 5% (V/V) autologous plasma containing 700 U/μL IL-2. The collected cell suspension was transferred to a new anti-CD16 antibody-unimmobilized flask and then cultured again for 21 days in a 5% CO₂ incubator set to 37° C. The OpTmizer medium containing 5% (V/V) autologous plasma was replaced by a fresh one every 2 to 4 days. In this way, the NK cell-enriched blood preparation of the second aspect of the present invention was prepared.

For actually using the NK cell-enriched blood preparation, it is required to perform a contamination test or pretreatment. Hereinafter, their procedures will be described simply.

(5) (Contamination Test)

The presence or absence of endotoxin in the culture solution was confirmed using Limulus ES-II (Wako Pure Chemical Industries, Ltd.) according to the protocol supplied with the kit. Also, the presence or absence of bacteria or mold was confirmed by colony formation assay using an aliquot of the culture solution applied to an agar medium.

(6) Pretreatment of NK Cell-Enriched Blood Preparation

Three weeks after culture, the culture solution was transferred to a centrifuge tube and centrifuged at 1200 rpm for 10 minutes, and the supernatant was then discarded. The precipitates were suspended by the addition of 50 mL of PBS (−) and centrifuged again at 1200 rpm for 10 minutes, and the supernatant was then discarded. This operation was performed 3 repetitive times to remove the medium components. Finally, the residue was suspended in 70 mL of lactate Ringer solution. In this way, the NK cell-enriched blood preparation was obtained as the final product. The blood preparation had an NK cell growth rate of approximately 16000 times after 14 days and approximately 44000 times after 21 days. This was more than 4 times the NK cell growth rate obtained in the conventional method for producing an NK cell-enriched blood preparation using only an anti-CD16 antibody, IL-2 and OK432, both after 14 days and after 21 days.

### Example 2

### Growth Rate of NK Cell

In order to confirm that the method for producing an NK cell-enriched blood preparation according to the present invention did not require high-temperature stimulation, the growth rate of NK cells was examined.

(Method)

In this Example, two samples shown below were examined for the growth rate of NK cells, etc., in blood obtained from each healthy donor whose gave informed consent to compare results obtained about the method for producing an NK cell-enriched blood preparation of the present invention using NK cell growth-stimulating factors comprising an anti-CD16 antibody, an anti-CD137 antibody, OK432, and IL-2 with results obtained about totally the same method as the method for producing an NK cell-enriched blood preparation of the present invention except that the anti-CD137 antibody was not used in the NK cell growth-stimulating factors.

Sample a: The NK cell growth-stimulating factors used were 1 μg/mL anti-CD16 antibody, 0.01 KE/mL OK432, and 700 U/mL IL-2 (concentrations were all indicated by the final concentrations). The NK cell growth-stimulating factors in this sample correspond to growth-stimulating factors used in JP Patent No. 4275680.

Sample b: The NK cell growth-stimulating factors used were 1 μg/mL anti-CD16 antibody, 0.01 KE/mL OK432, a 1 μg/mL solution of an anti-CD137 antibody (4-1 BB, BioLegend), and 700 U/mL IL-2 (concentrations were all indicated by the final concentrations). The NK cell growth-stimulating factors in this sample correspond to the NK cell growth-stimulating factors of the present invention.

Unlike the production method according to JP Patent No. 4275680, the samples a and b do not undergo the high-temperature stimulation step at 39° C.

The basic operation of the method for producing an NK cell-enriched blood preparation was the same as in Example 1 except for difference in the composition of each sample described above and steps. The day when PBMCs were suspended at a cell density of 1×10⁶ cells/mL in OpTmizer medium was defined as day 0. The day when stimulation and culture were initiated by stimulation with each stimulating factor and addition of 10% autologous plasma to the medium was defined as day 0. At culture days 3, 5, 7, 10, 12, 14, 17, and 21, an aliquot of each culture solution was collected, and the total number of cells in the culture solution was determined.

NK cells in each culture solution were assayed using a flow cytometry analysis method at day 0, day 14 and day 21. Specifically, the NK cells in the blood preparation were immunostained using a combination of fluorescent material-labeled monoclonal antibodies (PC5- or ECD-labeled anti-CD3 antibody and PE- or PC5-labeled anti-CD56 antibody; Immunotech). The immunostaining was performed by adding, to the cell suspension, each antibody in an amount recommended by the document supplied with the antibody, and staining the cells at room temperature for 15 minutes in the dark, followed by centrifugation and washing off of the supernatant containing the fluorescently labeled antibodies. Subsequently, the kinetics of the NK cells were assayed by flow cytometry using Cytomics FC500 (Beckman Coulter, Inc.) based on the combination of the antibodies. The assay data was analyzed by CXP analysis.

(Results)

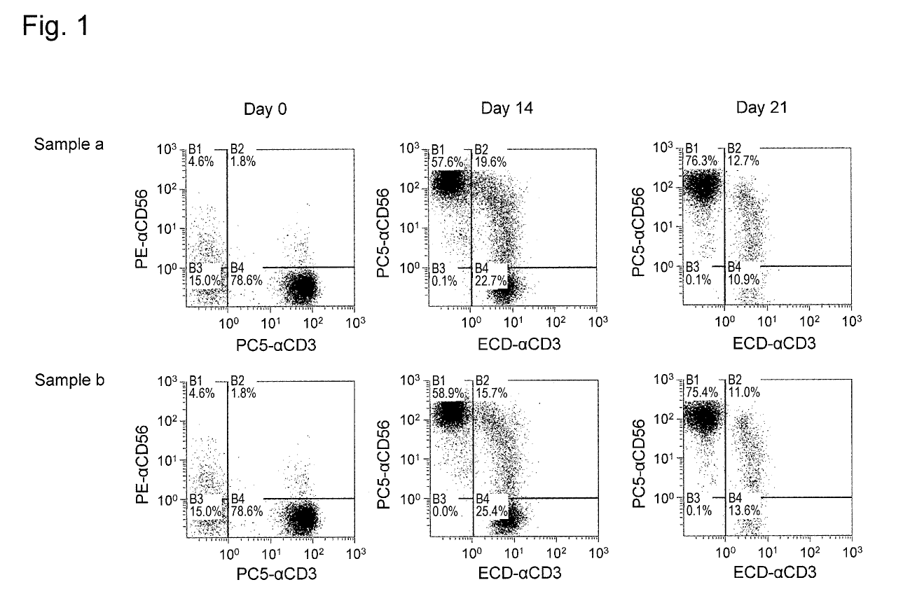

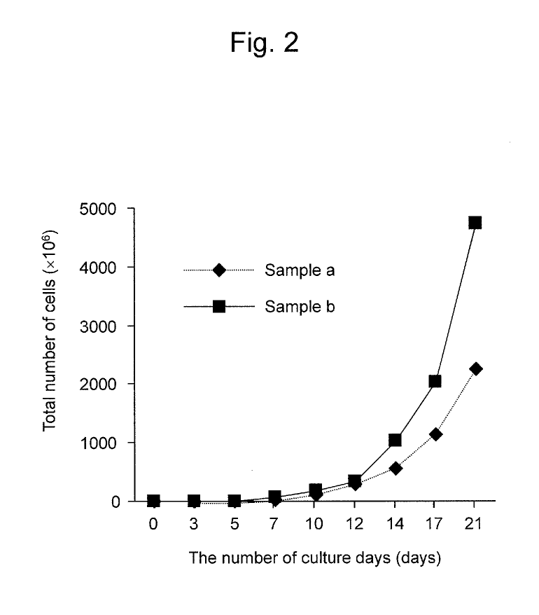

The results are shown in FIGS. 1 and 2, and Table 1.

TABLE 1

| | | The | | Absolute | |
|---|---|---|---|---|---|
| | | number | | number | Cytotoxic |
| Culture | | of cells | | of NK cells | activity % |
| days (days) | Sample | (×10⁶) | NK % | (×10⁶) | (E/T = 1.5/1) |
| 0 | a | 5.0 | 4.6 | 0.08 | — |
| | b | 5.0 | 4.6 | 0.08 | — |
| 14 | a | 555.9 | 57.6 | 320.2 | 76 |
| | b | 2265.6 | 58.9 | 1334.4 | 79 |
| 21 | a | 1036.3 | 76.3 | 790.7 | 72 |
| | b | 4723.6 | 75.4 | 3561.6 | 76 |

As shown in FIG. 1, no significant difference in % (B1; CD3⁻CD56⁺) of NK cells in the total number of cells was observed between samples a and b at all of culture days 0, 14, and 21.

As shown in FIG. 2, however, the total number of cells started to differ between samples a and b from around day 13 after the start of culture. In response to the results, the absolute number of NK cells in sample b reached, as shown in Table 1, approximately 4 times the absolute number of NK cells in sample a at day 14. The NK cell-enriched blood preparation used in this aspect was found to have the number of NK cells 10000 times or more the average number of each blood cell per unit volume from (Total number of cells at the completion of culture x % of NK cells thereto)/(The number of PBMCs at the start of culture x % of NK cells thereto), when this solution was compared in the same amount as the amount of blood used in the culture. There results demonstrated that the production method of the present invention can more efficiently grow NK cells than the conventional method without requiring high-temperature stimulation.

### Example 3

### Assay of Activated NK Cell

The activation of NK cells in the NK cell-enriched blood preparation of the present invention was assayed on the basis of cytotoxic activity against a K562 cell line, which was targeted by NK cells.

(Method)

First, leukemia cell line K562 cells were labeled with a fluorescent dye Calcein-AM. The labeling was performed by incubation at 37° C. for 30 minutes in a RPMI-1640 medium (containing 10% fetal bovine serum) supplemented with a 1/100 volume of Calcein-AM solution (Dojindo Laboratories). The cells thus labeled were washed with PBS (−) and used as target cells. Next, NK cells in the sample a-, and b-derived NK cell-enriched blood preparations produced by the method of Example 2 were separately used as effector cells (E). These effector cells were adjusted to their respective predetermined values in terms of the ratio (E/T ratio) to the target K562 cells (target cells: T), then separately placed in a 96-well plate, and reacted at 37° C. for 2 hours at a CO₂ concentration of 5%. After the reaction, the amounts of the target cells that retained fluorescence, i.e., survived, were detected on the basis of their fluorescence intensities using Terascan VP (Minerva Tech K.K.). The value of cytotoxic activity against K562 was calculated by comparison with a control before cytotoxicity, i.e., fluorescence intensity from a state nonsupplemented with effector cells.

(Results)

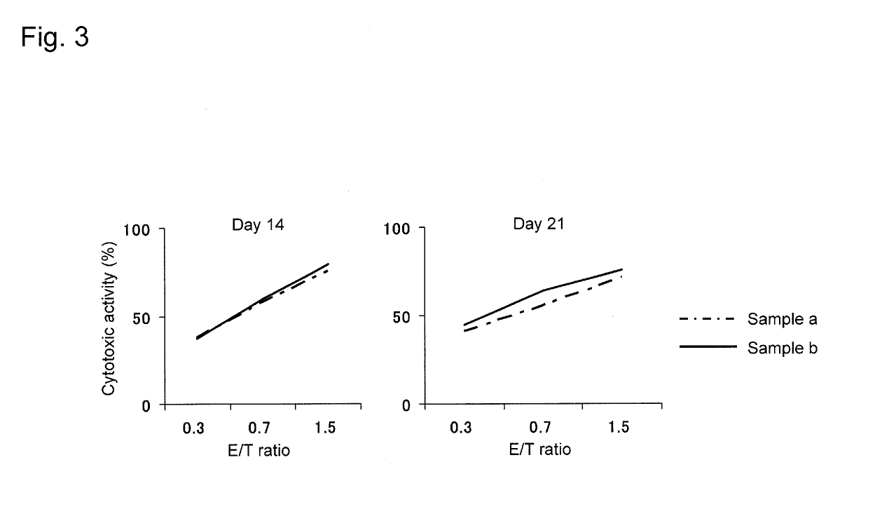

The cytotoxic activity of NK cells derived from samples a and b produced by the method of Example 2 is shown in Table 1 above, and the cytotoxic activity of NK cells derived from samples a and b at day 14 and day 21 is shown in FIG. 3. The E/T ratios used are shown in the corresponding tables or diagrams.

As is evident from Table 1 and FIG. 3, the NK cells obtained in the method for producing an NK cell-enriched blood preparation according to the present invention had cytotoxic activity substantially equivalent to that of the method for producing an NK cell-enriched blood preparation according to JP Patent No. 4275680. In addition, the cytotoxic activity of the NK cells was not reduced even after culture until day 21. These results demonstrated that the method for producing an NK cell-enriched blood preparation according to the present invention can provide the cytotoxic activity of NK cells as high as that obtained by the production method according to JP Patent No. 4275680, and can also yield a larger number of activated NK cells.

### Example 4

### Method for Producing Cancer Patient-Derived NK Cell-Enriched Blood Preparation, Growth Rate of NK Cell, and Activity Measurement of NK Cell

In Example 1 above, the blood donor used for the production of the NK cell-enriched blood preparation was a healthy individual. In this Example, a cancer patient, who is an actual individual to be treated, was used as a blood donor and examined for whether an NK cell-enriched blood preparation produced by the method of the present invention using blood derived from the cancer patient could also permit efficient growth of NK cells.

### (1) Method for Producing NK Cell-Enriched Blood Preparation

The basic method followed the method described in Example 1. However, three cancer patients shown in Table 2 who gave informed consent were used as donors.

TABLE 2

| Patient | | | |
|---|---|---|---|
| No. | Age (years old) | Sex | Type of cancer, etc. |
| 1 | 73 | Male | Esophageal cancer: postoperative |
| | | | recurrence and metastasis |
| 2 | 66 | Female | Breast cancer: stage IV |
| 3 | 62 | Male | Bile duct cancer: stage IV |

### (2) Growth Rate of NK Cell

The basic method for producing an NK cell-enriched blood preparation followed the method described in Example 1. A negative control sample (sample A) without the addition of an anti-human CD137 antibody to NK cell growth-stimulating factors in the stimulation step to examine the growth rate of NK cells, and a sample (sample B) treated with the NK cell growth-stimulating factors of the present invention also including the anti-human CD137 antibody were prepared according to the method described in Example 2. The number of culture days in the culture step was set to 20 days for the sample derived from patient No. 1, 21 days for the sample derived from patient No. 2, and 14 days for the sample derived from patient No. 3.

The total number of cells in a culture solution and the absolute number of NK cells were measured according to the method described in Example 2.

### (3) Activity Measurement of NK Cell

The basic method followed the method described in Example 3. After reaction, the amount of fluorescence released into a supernatant was detected using a multi-label reader (PerkinElmer, Inc.). The value of cytotoxic activity against K562 was calculated by comparison with fluorescence intensity from a state nonsupplemented with effector cells.

(Results)

The results are shown in Table 3.

TABLE 3
At start of culture (day 0)

| | The number | | Absolute number |
|---|---|---|---|
| | of PBMCs | | of NK cells |
| Patient | (×10⁶) | NK % | (×10⁶) |
| #01 | 27.2 | 4.8 | 1.3 |
| #02 | 20.0 | 7.0 | 1.4 |
| #03 | 13.4 | 4.5 | 0.6 |
| Average | 20.2 | 5.4 | 1.1 |
| STDEV | 6.9 | 1.4 | 0.4 |

Sample A (at the end)

| | | Total number of | | | | |
|---|---|---|---|---|---|---|
| | | cells at | | Absolute | Folds of | Cytotoxic |
| | Culture | completion of | | number of NK | NK cell | activity % |
| Patient | days | culture (×10⁶) | NK % | cells (×10⁶) | expansion | (E/T = 1.2/1) |
| #01 | 20 | 1886.4 | 76.3 | 1439.3 | 1107 | 53.2 |
| #02 | 21 | 3444.0 | 39.9 | 1374.2 | 982 | 20.2 |
| #03 | 14 | 664.0 | 70.4 | 467.5 | 779 | 52.8 |
| Average | | 1998.1 | 62.2 | 1093.6 | 955.9 | 42.1 |
| STDEV | | 1393.4 | 19.5 | 543.3 | 165.5 | 18.9 |

Sample B (at the end)

| | | Total number of | | Absolute | Folds of | Cytotoxic |
|---|---|---|---|---|---|---|
| | Culture | cells cultured | | number of NK | NK cell | activity % |
| Patient | days | (×10⁶) | NK % | cells (×10⁶) | expansion | (E/T = 1.2/1) |
| #01 | 20 | 1841.6 | 85.7 | 1578.3 | 1214 | 69.2 |
| #02 | 21 | 3388.0 | 43.8 | 1483.9 | 1060 | 37.2 |
| #03 | 14 | 848.0 | 74.8 | 634.3 | 1057 | 48.3 |
| Average | | 2025.9 | 68.1 | 1232.2 | 1110.4 | 49.5 |
| STDEV | | 1280.0 | 21.7 | 519.9 | 89.8 | 12.9 |

As shown in Table 3, no significant difference in % of NK cells in the total number of cells was observed between samples A and B. However, the absolute number of NK cells was increased in sample B compared with sample A. These results demonstrated that the production method of the present invention can more efficiently grow NK cells than the conventional method, even if cancer patient-derived blood is used.

These results also demonstrated that the NK cell-enriched blood preparation obtained by the production method of the present invention has cytotoxic activity comparable to that of an NK cell-enriched blood preparation obtained by the conventional production method that does not involve an anti-CD137 antibody, even if cancer patient-derived blood is used.

### Example 5

### Method for Producing NK Cell-Enriched Blood Preparation (2)

The method for producing an NK cell-enriched blood preparation of the present invention described in Example 1 was examined for an NK cell growth effect by stimulation with NK cell growth-stimulating factors different from those of Example 1.

### (1) Method for Producing NK Cell-Enriched Blood Preparation

The basic method followed the method described in Example 1. However, the blood donor was a 75-year-old female pancreatic body cancer patient (stage IV) who gave informed content, and treatment as described below was performed in the stimulation step.

### (Sample α)

The stimulation step using the NK cell growth-stimulating factors described in Example 1 was performed.

### (Sample β)

0.2 mg of an anti-CD16 antibody (Clone 3 GB, Beckman Coulter, Inc.) was dissolved in 1 mL of sterile distilled water, and this solution was sterilized by filtration through a 0.22 μm filter. The solution was adjusted to 1 μg/mL in terms of the final concentration by the addition of 199 mL of sterile distilled water, and then mixed. After filtration, 5 mL of the anti-CD16 antibody solution was placed in a 25 cm² culture flask and left standing overnight at 37° C. to immobilize the anti-CD16 antibody in this solution onto the inner wall of the flask. Then, the solution was discarded, and the inside of the flask was washed twice with sterile PBS (−).

4.7 mL of a suspension of PBMCs prepared in the same way as in Example 1 was transferred into the flask. Subsequently, 4.7 μL of a 1000-fold diluted solution of an anti-CD3 antibody (Orthoclone OKT3, Janssen Pharmaceutical K.K.), 24 μL of 0.2 μg/μL solution of an anti-human CD137 antibody (4-1BB, BioLegend, Inc.), 19 μL of 4 μL/mL (final concentration) aqueous OK432 (Picibanil; Chugai Pharmaceutical Co., Ltd.) solution, 0.5 μM/mL bisphosphonate derivative (zoledronic acid; trade name: Zometa (registered trademark), Novartis Pharma K.K.), and 3.7 μL of 900 U/μL IL-2 (Proleukin; Chiron Corp.) solution were added to the suspension of PBMCs, and the mixture was sufficiently stirred.

The number of culture days in the culture step was set to 15 days.

### (2) Growth Rate of NK Cell and Activity Measurement of NK Cell

The total number of cells and the absolute number of NK cells in the culture solution were measured according to the method described in Example 2 at day 3, day 5, day 6, day 8, day 10, day 12, and day 15 after culture. The activity measurement of NK cells was performed according to the method described in Example 4.

### (3) The Number of γδT Cell or Growth Rate of αβT Cell

The absolute numbers of γδT cells and αβT cells were determined using a flow cytometry analysis method as in the NK cells of Example 2. The γδT cells and the αβT cells in the blood preparation were immunostained using a combination of fluorescent material-labeled monoclonal antibodies (FITC-labeled anti-Vγ9 antibody and ECD-labeled anti-CD3 antibody; Immunotech). The immunostaining was performed by adding, to the cell suspension, each antibody in an amount recommended by the document supplied with the antibody, and staining the cells at room temperature for 15 minutes in the dark, followed by centrifugation and washing off of the supernatant containing the fluorescently labeled antibodies. Subsequently, the kinetics of the γδT cells and the αβT cells were assayed by flow cytometry using Cytomics FC500 (Beckman Coulter, Inc.) based on the combination of the antibodies. The assay data was analyzed by CXP analysis. In general, γδT cells are distributed in zone CD3⁺Vγ9⁺; cells (including NK cells) other than T cells are distributed in zone CD3⁻Vγ9⁻; and αβT cells are distributed in zone CD3⁺Vγ9⁻.

(Results)

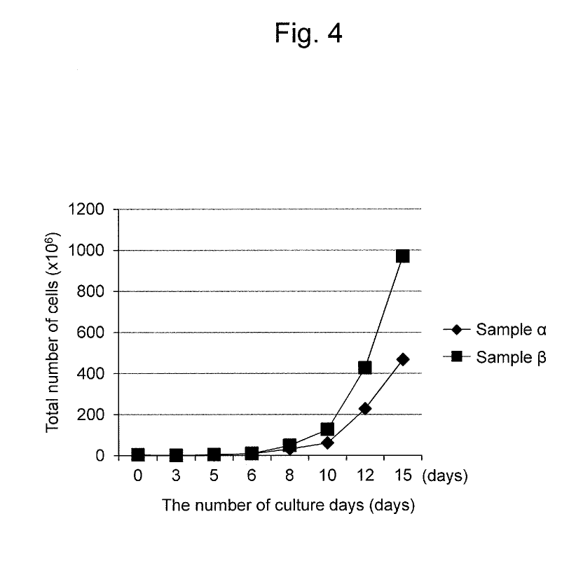

The results are shown in FIG. 4 and Table 4.

TABLE 4
At start of culture (day 0)

| | | | Absolute | | Absolute | | Absolute |
|---|---|---|---|---|---|---|---|
| | The number | | number of | | number of | | number of |
| Donor | of PBMCs | | NK cells | | γδT cells | | αβT cells |
| patient | (×10⁶) | NK % | (×10⁶) | γδT % | (×10⁶) | αβT % | (×10⁶) |
| | 4.7 | 14.5 | 0.7 | 2.4 | 0.11 | 65.0 | 3.1 |

At the end of culture (day 15)

| | | | Absolute | | |
|---|---|---|---|---|---|
| | Total number | | number of | Folds of | Cytotoxic |
| | of cells | | NK cells | NK cell | activity % |
| Sample | cultured (×10⁶) | NK % | (×10⁶) | expansion | (E/T = 1.2/1) |
| α | 468.0 | 87.2 | 408.1 | 599 | 70.3% |
| β | 972.0 | 79.5 | 772.7 | 1134 | 77.3% |

At the end of culture (day 15)

| | Total | | Absolute | | | Absolute | | |
|---|---|---|---|---|---|---|---|---|
| | number of | | number | | | number | | |
| | cells | | of γδT | Folds of | | of αβT | Folds of | |
| | cultured | | cells | γδT cell | | cells | αβT cell | NK+ |
| Sample | (×10⁶) | γδT % | (×10⁶) | expansion | αβT % | (×10⁶) | expansion | γδT % |
| α | 468.0 | 5.6 | 26.2 | 232 | 6.8 | 31.8 | 10 | 92.8% |
| β | 972.0 | 15.5 | 150.7 | 1336 | 4.7 | 45.7 | 15 | 95.0% |

As shown in FIG. 4, no significant different in the total number of cells was observed between samples α and β up to culture day 6. However, upon further culture, cells in sample β stimulated with NK cell growth-stimulating factors supplemented with an anti-CD3 antibody and a bisphosphonate derivative were more efficiently grown than those in sample α, and the total number of cells in sample β reached, as shown in FIG. 4 and Table 4, approximately twice the total number of cells in sample α at day 15 (at the completion of culture). Sample β also had approximately twice the absolute number of NK cells in sample α. Meanwhile, the NK cells of sample β had cytotoxic activity equivalent to that of NK cells of sample α.

As for the growth rates of γδT cells and αβT cells between samples α and β, sample β had approximately 6-fold and approximately 1.5-fold rises in the growth rates of γδT cells and αβT cells, respectively, compared with sample α.

The results described above demonstrated that NK cells as well as γδT cells and αβT cells can be grown further efficiently by further adding an anti-CD3 antibody and a bisphosphonate derivative, etc. to NK cell growth-stimulating factors including an anti-CD16 antibody, OK432, an anti-CD137 antibody, and a cytokine.

All publications, patents, and patent applications cited herein are incorporated herein by reference in their entirety.

1. A method for producing an NK cell-enriched blood preparation, comprising:
a stimulation step of stimulating NK cells comprised in blood collected from an organism, with NK cell growth-stimulating factors comprising an anti-CD16 antibody, OK432, an anti-CD137 antibody, and a cytokine; and
a culture step of culturing the blood at a physiological cell temperature after the stimulation step.

2. The production method according to claim 1, wherein the cytokine is IL-2.

3. The production method according to claim 1, wherein the NK cell growth-stimulating factors further comprise an anti-CD3 antibody and/or a bisphosphonate derivative or a salt thereof, or a hydrate thereof.

4. The production method according to claim 1, wherein the physiological cell temperature is 36.5 to 37.5° C.

5. The production method according to claim 1, wherein the culture period in the culture step is 7 days to 30 days.

6. The production method according to claim 1, wherein the anti-CD16 antibody is immobilized on a solid-phase support.

7. An NK cell-enriched blood preparation obtained by a production method according to claim 1.

8. A composition for NK cell enrichment comprising an anti-CD16 antibody, OK432, an anti-CD137 antibody, and a cytokine.

9. The composition according to claim 8, wherein the cytokine is IL-2.

10. The composition according to claim 8, further comprising an anti-CD3 antibody and/or a bisphosphonate derivative or a salt thereof, or a hydrate thereof.

11. A kit for production of NK cell-enriched blood comprising a composition for NK cell enrichment according to claim 8.
